SEQUENCE LISTING (5 sequences)

SEQ ID NO: 1 - protein, 142 aa, Homo sapiens
  misc_feature: Human LALBA Amino Acid Precursor Sequence
MRFFVPLFLVGILFPAILAKQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL

SEQ ID NO: 2 - DNA, 742 bp, Homo sapiens
  misc_feature: Human LALBA cDNA Sequence
atttcaggttcttgggggtagccaaaatgaggttctttgtccctctgttcctggtgggcatcctgttccctgccatcctggccaagcaattcacaaaatgtgagctgtcccagctgctgaaagacatagatggttatggaggcatcgctttgcctgaattgatctgtaccatgtttcacaccagtggttatgacacacaagccatagttgaaaacaatgaaagcacggaatatggactcttccagatcagtaataagctttggtgcaagagcagccaggtccctcagtcaaggaacatctgtgacatctcctgtgacaagttcctggatgatgacattactgatgacataatgtgtgccaagaagatcctggatattaaaggaattgactactggttggcccataaagccctctgcactgagaagctggaacagtggctttgtgagaagttgtgagtgtctgctgtccttggcacccctgcccactccacactcctggaatacctcttccctaatgccacctcagtttgtttctttctgttcccccaaagcttatctgtctctgagccttgggccctgtagtgacatcaccgaattcttgaagactattttccagggatgcctgagtggtgcactgagctctagacccttactcagtgccttcgatggcactttcactacagcacagatttcacctctgtcttgaataaaggtcccactttgaagtcaaaaaaaaaaaaaaa

SEQ ID NO: 3 - protein, 143 aa, Mus musculus
  misc_feature: Mouse LALBA Amino Acid Sequence
MMHFVPLFLVCILSLPAFQATELTKCKVSHAIKDIDGYQGISLLEWACVLFHTSGYDTQAVVNDNGSTEYGLFQISDRFWCKSSEFPESENICGISCDKLLDDELDDDIACAKKILAIKGIDYWKAYKPMCSEKLEQWRCEKP

SEQ ID NO: 4 - DNA, 733 bp, Mus musculus
  misc_feature: Mouse LALBA cDNA Sequence
ggagcagtcaaaatgatgcatttcgttcctttgttcctggtgtgtattttgtcgttgcctgcctttcaagccacagagcttacaaaatgcaaggtgtcccatgccattaaagacatagatggctatcaaggcatctctttgcttgaatgggcctgtgttttatttcataccagtggctacgacacacaagctgttgtcaacgacaacggcagcacagagtacggactcttccagatcagtgacagattttggtgtaaaagtagtgagttccccgagtcggagaacatctgtggcatctcctgtgacaagttattggatgacgagttggatgatgacatagcgtgtgccaagaagatcctggctatcaaaggaatcgactactggaaagcctacaagcccatgtgctctgagaagcttgaacagtggcgttgtgagaagccctgagccccccccccccccccccccgtccttgctgctcctgccccgtggtcaggaatgcctcttccctaaggctacctcagcttggctcttgctattcctgtgaagatgatctgcctctgagccttgtaccctgtagtgacaccaccggactctagaggacttttttttccctatgggagtgtgactggcgcactggactgcaaacccttgcttagtgacggcgagggtctcgatgggggttttacaaaatcgagagagccctctcctgtcccaaataaagggccagacttga

SEQ ID NO: 5 - protein, 123 aa, Homo sapiens
  misc_feature: Human LALBA Amino Acid Sequence
KQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL

(19) United States
(12) Patent Application Publication
(10) Pub. No.: US 2021/0106664 A1
(43) Pub. Date: Apr. 15, 2021

(54) ALPHA-LACTALBUMIN VACCINATION FOR INHIBITING GROWTH AND DEVELOPMENT OF MALE BREAST CANCERS

(71) Applicant: Cleveland Clinic Foundation, Cleveland, OH (US)

(72) Inventor: Vincent K. TUOHY, Broadview Heights, OH (US)

(21) Appl. No.: 17/035,574

(22) Filed: Sep. 28, 2020

Related U.S. Application Data
(60) Provisional application No. 62/907,219, filed on Sep. 27, 2019.

(51) Int. Cl. A61K 39/00; A61P 35/00

(52) U.S. Cl. CPC A61K 39/0011; A61K 2039/812; A61K 2039/55; A61P 35/00

(57) ABSTRACT

Methods comprising a step of administering, to a mammalian male subject, a composition comprising α-lactalbumin for the treatment of male breast cancer.

### CROSS REFERENCE TO RELATED APPLICATION

This application claims the benefit of priority to U.S. Provisional Application No. 62/907,219, filed Sep. 27, 2019, which is incorporated by reference herewith in its entirety.

### BACKGROUND

Male breast cancer accounts for approximately 1% or less of breast cancer cases worldwide. In the United States, an estimated 2,670 men were expected to be diagnosed with breast cancer in 2019. Treatment options typically include surgery (often mastectomy), radiation therapy, chemotherapy, and hormone therapy. Not all patients with male breast cancer are responsive to each kind of treatment. For example, hormone therapy can only be effective for the subset of breast cancers that need hormones to grow. Moreover, because of the rarity of breast cancer in men, male breast cancer is typically diagnosed at a late stage, making treatment challenging.

There is a need for prophylactic and therapeutic methods for reducing the risk of and treating, respective, male breast cancer.

### SUMMARY

The present disclosure provides, among other things, methods useful in the prevention or treatment of cancers, including male breast cancer, by immunization using immunodominant tissue specific proteins or fragments thereof.

In one aspect, the methods are useful in the prevention and treatment of male breast cancer.

In one aspect, provided are methods comprising the step of: administering, to a mammalian male subject, a composition comprising an a-lactalbumin polypeptide or an immunogenic fragment thereof.

In some embodiments, the mammalian male subject is identified as being at risk of developing breast cancer. For example, in some embodiments, the mammalian male subject has one or more risk factors selected from the group consisting of older age, family history of breast cancer, high estrogen level, exposure to estrogen, lower androgen level, Klinefelter's syndrome, liver disease, obesity, testicle disease or surgery, gynecomastia, prolactinoma, radiation exposure to the chest, a genotype associated with breast cancer, or a gene expression profile associated with breast cancer.

In some embodiments, the mammalian male subject has one or more signs or symptoms of breast cancer.

In some embodiments, the mammalian male subject is diagnosed with breast cancer. In some embodiments, the mammalian male subject has a breast carcinoma. In some embodiments, the breast carcinoma expresses α-lactalbumin. In some embodiments, the breast carcinoma exhibits increased expression of α-lactalbumin as compared to a reference level, e.g., at least 2-fold, at least 5-fold, at least 10-fold, or at least 20-fold higher than the reference level.

In some embodiments, the mammalian male subject is a human.

In some embodiments, the composition comprises an amount of an α-lactalbumin polypeptide or an immunogenic fragment thereof effective to induce an immune response against α-lactalbumin. In some embodiments, the immune response comprises T-cells specific for α-lactalbumin. In some embodiments, the immune response comprises T-cells producing interferon gamma (IFNγ). In some embodiments, the immune response comprises CD4+ T-cells, CD8+ T-cells, or both CD4+ T-cells and CD8+ T-cells.

In some embodiments, the immune response comprises immunoglobulin-expressing cells specific for α-lactalbumin. In some embodiments, the immunoglobulin-expressing cells comprise B-cells.

In some embodiments, the composition further comprises an adjuvant.

In some embodiments, the α-lactalbumin is human α-lactalbumin.

In some embodiments, the composition is administered systemically.

In some embodiments, the method further comprises administering to the mammalian male subject one or more booster compositions comprising an α-lactalbumin polypeptide or an immunogenic fragment thereof.

### BRIEF DESCRIPTION OF THE DRAWINGS

FIGS. 1A-1C Show α-Lactalbumin Gene Expression in TNBC Compared to All Other Invasive Breast Cancers. An ONCOMINETM data base search of the Cancer Genome Atlas (TCGA) Breast shows a highly significant increased expression of the α-lactalbumin gene (LALBA) in triple-negative breast cancer (TNBC) compared to all other invasive ductal breast carcinomas with identifiable biomarkers (P<0.0001). This can be seen using (FIG. 1A) column charts, (FIG. 1B) heat maps, or (FIG. 1C) box charts. Heat map colors are z-score normalized to depict relative values within the row.

FIGS. 2A and 2B show results of α-Lactalbumin Gene and Protein Expression in Human TNBC. FIG. 2A provides the results of RT-PCR was performed on RNA extracted from formalin-fixed paraffin embedded TNBC tissues and the amplified products were visualized on an agarose gel. Lane 1 (upper gel) shows a DNA ladder. Lanes 2-6 show amplification from human lactating adenomas (LA) serving as positive controls. Lanes 7-17 show amplification from human TNBC primary tumors. β-Actin gene expression served as an internal control (lower gel). FIG. 2B presents immunohistochemical analysis of formalin-fixed paraffin embedded human TNBC tissue sections show positive staining in 5/6 primary human TNBC tumors examined with TNBC-12 being the only TNBC tumor that did not show α-lactalbumin gene expression and protein detection. Arrows point to the unstained stromal cells.

FIG. 3 shows levels of α-Lactalbumin Gene Expression in Male Breast Carcinoma Compared to Normal Breast Tissue. An ONCOMINETM data base search of TCGA Breast shows a highly significant increased expression of the α-lactalbumin gene (LALBA) in male breast cancers compared to normal breast tissues (P<0.02).

FIG. 4. Comparing the Lethality of Female Cancers. The lethality of each human gynecologic malignancy was determined by dividing the annual number of deaths by the annual number of diagnosed cases to yield the mortality-incidence ratio (MIR).

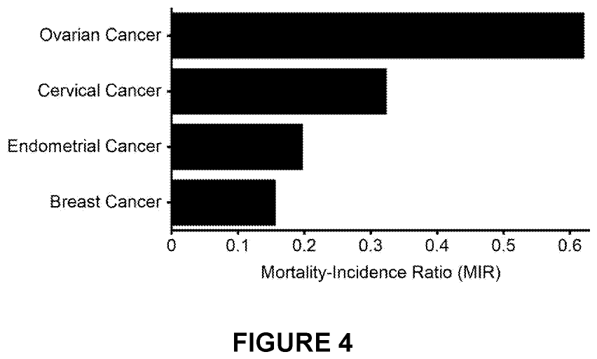

### DETAILED DESCRIPTION

### Definitions

As used herein, “adjuvant” means a substance, which when administered before, together with, or after administration of an antigen, accelerates, prolong and/or enhances the quality and/or strength of an immune response to the antigen in comparison to the response elicited by administration of the antigen alone. In some embodiments, the adjuvant comprises a mixture of at least two polysaccharides and a metabolizable oil. In some such embodiments, the mixture of at least two polysaccharides comprises 1) a glucan (e.g., β-glucan) and 2) another polysaccharide or polysaccharide mixture (e.g., a mixture of chitins, glucans, and mannans; e.g., zymosan).

As used herein, the term “antigen” has its ordinary meaning in the art and refers to any molecule or portion of a molecule that can, either by itself or in conjunction with an adjuvant and/or pharmaceutically acceptable carrier, generate an immune response, e.g., an antibody and/or T-cell response.

The term “immune response” refers herein to any response to an antigen or antigenic determinant by the immune system. Exemplary immune responses include humoral immune responses (e.g. production of antigen-specific antibodies (neutralizing or otherwise)) and cell-mediated immune responses (e.g. lymphocyte proliferation). Type-1 proinflammatory immune responses are characterized by the production of IFNγ. Type-2 regulatory immune responses are characterized by expression of IL-4 or IL-5. Type-17 proinflammatory immune responses are characterized by expression of IL-17. In some instances, a mixed immune response (e.g., both a Type-1 and a Type-17 response) can be generated.

As used herein, “percent identity” or “percentage identity” between amino acid or nucleotide sequences is synonymous with “percent homology,” and which can be determined, for example, using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993). The noted algorithm is incorporated into the NBLAST and XBLAST programs of Altschul et al. (J. Mol. Biol. 215, 403-410, 1990).

The phrase “pharmaceutically acceptable carrier” as used herein means a pharmaceutically acceptable material, composition, or vehicle, such as a liquid or solid filler, diluent, excipient, thickening agent, solvent, or encapsulating material, involved in carrying or transporting the subject compound from one organ, or portion of the body, to another organ, or portion of the body. The term “carrier” encompasses both carriers that are not covalently attached and those that are covalently attached to the compounds or compositions they transport.

As used herein, the terms “polypeptide” and “protein” are used interchangeably and generally have their art-recognized meaning of a polymer of at least three amino acids. The term “polypeptide” can refer to polypeptides in their neutral (uncharged) forms or as salts, and either unmodified or modified, e.g., by glycosylation, side chain oxidation, or phosphorylation. The term “polypeptide” can also be used to refer to specific functional classes of polypeptides. When used to refer to a functional class of polypeptides, the term is intended to include functional fragments, variants (e.g., allelic variants), and derivatives of a reference polypeptide, as well as the full length, wild type version of the reference polypeptide. In some embodiments, a polypeptide of a certain functional class shares at least 50%, at least 55%, at least 60%, at least 65%, at least 70%, at least 75%, at least 80%, at least 85%, at least 90%, at least 92.5%, at least 95%, at least 97.5%, or at least 99% sequence identity at the amino acid level with the full-length version of a reference polypeptide. For example, “α-lactalbumin polypeptides,” as used herein, includes α-lactalbumin as well as polypeptides having an amino acid sequence having sufficient sequence identity with the amino acid sequence of α-lactalbumin (or a portion thereof) to elicit an α-lactalbumin-specific immune response.

As used herein, the terms “subject” and “patient” are interchangeable and refer to an organism that receives a treatment or vaccine (e.g., by being administered a composition or formulation as disclosed herein). Examples of subjects and patients include mammals, such as humans or non-human animals.

The phrases “therapeutically-effective amount” and “effective amount” as used herein means the amount of an agent that is effective for producing a desired effect (e.g., a therapeutic effect, or a response such as an immune response) in a subject or in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a composition, such that at least one symptom of the disease is decreased or prevented from worsening.

The term “reference” refers to any sample, standard, or level that is used for comparison purposes. The phrases “reference standard” and “reference level” may be used interchangeably. In some embodiments, “reference level” refers to a value or number derived from a reference sample, subject, or population of subjects (e.g., an average value). In some embodiments, the sample or subject from whom the reference level is derived is matched to a sample of a subject by at least one of the following criteria: age, sex, weight, disease stage, and overall health. For example, in some embodiments, a reference level is obtained from normal (e.g., noncancerous) tissue, e.g., normal breast tissue. In some embodiments, the reference level is zero.

The term “retired self-protein” refers to a self-protein that is no longer expressed in normal aged tissues at autoimmunogenic levels. The term “retired self-antigen” refers to an antigen from a retired self-protein. In some embodiments, the retired self-antigen comprises a fragment of the retired self-protein. In some embodiments, the retired self-antigen comprises a full-length version of the retired self-protein.

### I. Methods

In one aspect, provided are methods comprising the step of administering, to a mammalian male subject, an amount of a composition comprising α-lactalbumin polypeptide or an immunogenic fragment thereof (e.g., as described further herein).

These methods may be useful, e.g,. for inducing or enhancing an immune response against α-lactalbumin, or for treating or ameliorating breast cancer in the subject.

In some embodiments, the mammalian male subject is administered the composition more than once, e.g., the mammalian male subject is additionally administered one or more boosters of a composition comprising an α-lactalbumin polypeptide or an immunogenic fragment thereof.

In some embodiments, the composition is administered by a systemic route, e.g., intravenously.

Subjects

In some embodiments, the mammalian male subject needing therapeutic or prophylactic treatment for breast cancer is is a human.

In some embodiments, the mammalian male subject is identified as being at risk of developing breast cancer. For example, the mammalian male subject may have one or more of the characteristics that indicate risk of developing breast cancer. Risk factors for male breast cancer include, but are not limted to, older age (e.g., in an adult male, a male or female older than 50, older than 55, older than 60, or older than 65), family history of breast cancer, high estrogen level, exposure to estrogen (e.g., through a hormone therapy), lower androgen level, Klinefelter's syndrome, liver disease, obesity, testicle disease or surgery, gynecomastia, prolactinoma, radiation exposure to the chest, genotype associated with cancer (e.g., mutations or polymorphisms at one more loci associated with breast cancer, e.g., HER2, BRCA1, and/or BRCA2), or gene expression profile associated with breast cancer.

An estimated risk value can be calculated for a given subject based on any or any combination of known risk factors. In some embodiments, the mammalian male subject has an estimated risk value falling above a reference threshold.

In some embodiments, the mammalian male subject exhibits one or more signs or symptoms of breast cancer. Examples of signs and symptoms of male breast cancer include, but are not limited to, a lump or thickening of breast tissue, skin changes to breast tissue (e.g., dimpling, puckering, redness, or scaling), changes to one or more nipples (e.g., redness, scaling, or turning inward), or nipple discharge)), and changes to hormone levels (e.g., increased estrogen levels or decreased levels of testosterone).

In some embodiments, the mammalian male subject is diagnosed with breast cancer. In some embodiments, the diagnosis is confirmed by imaging and/or biopsy.

In some embodiments, the mammalian male subject has a breast carcinoma. In some embodiments, the breast carcinoma expresses α-lactalbumin, e.g., increased expression of α-lactalbumin relative to a reference level.

In some embodiments, a tissue sample (which can be a solid and/or liquid tissue sample) from the mammalian male subject exhibits expression of α-lactalbumin, e.g., increased expression of α-lactalbumin relative to a reference level.

An increased level of α-lactalbumin relative to a reference level may be, e.g., at least 2-fold, at least 5-fold, at least 10-fold, at least 15-fold, at least 20-fold, or at least 25-fold greater than the reference level.

### II. Compositions

α-lactalbumin Polypeptides

In some embodiments, the composition comprises an α-lactalbumin polypeptide (e.g., a human α-lactalbumin polypeptide) or an immunogenic fragment thereof. In some embodiments, the composition comprises multiple (e.g., at least 2, 3, 4, 5, 6, 7, 8, 9, or 10) different α-lactalbumin polypeptides or fragments. In some embodiments, one or more fragments are linked. In some embodiments the one or more fragments are linked to make a multivalent immunogen. In some embodiments, the fragments are linked via a linker molecule. In some embodiments, the composition comprises a nucleic acid encoding an α-lactalbumin polypeptide (or immunogenic fragment thereof) instead of or in addition to the α-lactalbumin polypeptide (or immunogenic fragment thereof).

The LALBA gene encodes α-lactalbumin, a principal protein of milk. α-lactalbumin forms the regulatory subunit of the lactose synthase (LS) heterodimer, and β1,4-galactosyltransferase (β4Gal-T1) forms the catalytic component. Together, these proteins enable LS to produce lactose by transferring galactose moieties to glucose. As a monomer, α-lactalbumin strongly binds calcium and zinc ions and may possess bactericidal or antitumor activity. The human LALBA gene contains 5 exons.

Human α-lactalbumin precursor protein has 142 amino acids and a molecular mass of 14,178 Da, and human α-lactalbumin has 123 amino acids.

In some embodiments, the α-lactalbumin polypeptide has 123 amino acids. The term “α-lactalbumin polypeptide” is intended to include fragments, variants (e.g., allelic variants), and derivatives thereof. Representative human α-lactalbumin cDNA and human α-lactalbumin protein sequences are well known in the art and are publicly available from the National Center for Biotechnology Information (NCBI). For example, at least one human UBE2D3 isoform is known. Human UBE2D3 isoform (NP_002280.1) is encoded by the transcript variant (NM_002289.2). Nucleic acid and polypeptide sequences of α-lactalbumin orthologs in organisms other than humans are well known and include, for example, chimpanzee α-lactalbumin (XM_016924811.2 and XP_016780300.1), monkey α-lactalbumin (XM_001102116.2 and XP 001102116.1), dog α-lactalbumin (NM_001003129.1 and NP_001003129.1), cattle α-lactalbumin (NM_174378.2 and NP_776803.1), mouse α-lactalbumin (NM_010679.1 and NP_034809.1), and rat α-lactalbumin (NM_012594.1 and NP_036726.1). Each of the above mRNA and protein sequences are hereby incorporated by reference. Representative sequences of α-lactalbumin orthologs are presented below in Table 1.

TABLE 1*
SEQ ID NO: 1 Human LALBA Amino Acid Precursor Sequence (NP_002280.1)

| | |
|---|---|
| 1 | mrffvplflv gilfpailak qftkcelsql lkdidgyggi alpelictmf htsgydtqai |
| 61 | vennesteyg lfqisnklwc kssqvpqsrn icdiscdkfl ddditddimc akkildikgi |
| 121 | dywlahkalc tekleqwlce kl |

SEQ ID NO: 2 Human LALBA cDNA Sequence (NM_002289.2; CDS: 27-455)

| | |
|---|---|
| 1 | atttcaggtt cttgggggta gccaaaatga ggttctttgt ccctctgttc ctggtgggca |
| 61 | tcctgttccc tgccatcctg gccaagcaat tcacaaaatg tgagctgtcc cagctgctga |
| 121 | aagacataga tggttatgga ggcatcgctt tgcctgaatt gatctgtacc atgtttcaca |
| 181 | ccagtggtta tgacacacaa gccatagttg aaaacaatga aagcacggaa tatggactct |
| 241 | tccagatcag taataagctt tggtgcaaga gcagccaggt ccctcagtca aggaacatct |
| 301 | gtgacatctc ctgtgacaag ttcctggatg atgacattac tgatgacata atgtgtgcca |
| 361 | agaagatcct ggatattaaa ggaattgact actggttggc ccataaagcc ctctgcactg |
| 421 | agaagctgga acagtggctt tgtgagaagt tgtgagtgtc tgctgtcctt ggcacccctg |
| 481 | cccactccac actcctggaa tacctcttcc ctaatgccac ctcagtttgt ttctttctgt |
| 541 | tcccccaaag cttatctgtc tctgagcctt gggccctgta gtgacatcac cgaattcttg |
| 601 | aagactattt tccagggatg cctgagtggt gcactgagct ctagaccctt actcagtgcc |
| 661 | ttcgatggca ctttcactac agcacagatt tcacctctgt cttgaataaa ggtcccactt |
| 721 | tgaagtcaaa aaaaaaaaaa aa |

SEQ ID NO: 3 Mouse LALBA Amino Acid Sequence (NP_034809.1)

| | |
|---|---|
| 1 | mmhfvplflv cilslpafga teltkckvsh aikdidgyqg isllewacvl fhtsgydtqa |
| 61 | vvndngstey glfgisdrfw ckssefpese nicgiscdkl lddeldddia cakkilaikg |
| 121 | idywkaykpm csekleqwrc ekp |

SEQ ID NO: 4 Mouse LALBA cDNA Sequence (NM_010679.1; CDS: 13-444)

| | |
|---|---|
| 1 | ggagcagtca aaatgatgca tttcgttcct ttgttcctgg tgtgtatttt gtcgttgcct |
| 61 | gcctttcaag ccacagagct tacaaaatgc aaggtgtccc atgccattaa agacatagat |
| 121 | ggctatcaag gcatctcttt gcttgaatgg gcctgtgttt tatttcatac cagtggctac |
| 181 | gacacacaag ctgttgtcaa cgacaacggc agcacagagt acggactctt ccagatcagt |
| 241 | gacagatttt ggtgtaaaag tagtgagttc cccgagtcgg agaacatctg tggcatctcc |
| 301 | tgtgacaagt tattggatga cgagttggat gatgacatag cgtgtgccaa gaagatcctg |
| 361 | gctatcaaag gaatcgacta ctggaaagcc tacaagccca tgtgctctga gaagcttgaa |
| 421 | cagtggcgtt gtgagaagcc ctgagccccc cccccccccc cccccgtcct tgctgctcct |
| 481 | gccccgtggt caggaatgcc tcttccctaa ggctacctca gcttggctct tgctattcct |
| 541 | gtgaagatga tctgcctctg agccttgtac cctgtagtga caccaccgga ctctagagga |
| 601 | cttttttttc cctatgggag tgtgactggc gcactggact gcaaaccctt gcttagtgac |
| 661 | ggcgagggtc tcgatggggg ttttacaaaa tcgagagagc cctctcctgt cccaaataaa |
| 721 | gggccagact tga |

SEQ ID NO: 5 Human LALBA Amino Acid Sequence

| | |
|---|---|
| 1 | kqftkcelsq llkdidgygg ialpelictm fhtsgydtqa ivennestey |
| 51 | glfgisnklw ckssqvpqsr nicdiscdkf lddditddim cakkildikg |
| 101 | idywlahkal ctekleqwlc ekl |

In some embodiments, compositions comprise an ortholog of LALBA, e.g., an ortholog of protein having an amino acid sequence of SEQ ID NO: 1, 3, or 5 or of a protein encoded by a nucleic acid having the nucleotide sequence of SEQ ID NO: 2 or 4.

In some embodiments, compositions comprise polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of SEQ ID NO: 1, 3, or 5, or a portion thereof.

In some embodiments, compositions comprise fusion polypeptides comprising an α-lactalbumin polypeptide (or immunogenic fragment thereof) and a heterologous polypeptide.

In certain embodiments, the α-lactalbumin polypeptide or immunogenic fragment thereof has an amino acid sequence that comprises at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, or 140 consecutive amino acids of an α-lactalbumin amino acid sequence (e.g., SEQ ID NO: 1, 3, or 5).

In some embodiments, compositions comprise an α-lactalbumin polypeptide or immunogenic fragment thereof that is a derivative, equivalent, variant, fragment, or mutant of α-lactalbumin.

In some embodiments, α-lactalbumin polypeptides are functional equivalents in that they have an amino acid sequence that is altered relative to the sequence of α-lactalbumin polypeptide (for example, by conservative substitution), yet still elicit immune responses. As used herein, the term “conservative substitution” denotes the replacement of an amino acid residue by another, biologically similar residue.

In some embodiments, compositions comprise nucleic acids, such as DNA molecules, encoding α-lactalbumin polypeptides (or immunogenic fragments thereof) described herein. In some embodiments, compositions comprise an expression vector comprising an open reading frame encoding an α-lactalbumin polypeptide or immunogenic fragment(s) thereof. In some embodiments, the α-lactalbumin nucleic acid includes regulatory elements that facilitate expression of the open reading frame. Such elements can include, for example, one or more of a promoter, an initiation codon, a stop codon, and a polyadenylation signal. One or more enhancers can be included. These elements can be operably linked to a sequence that encodes the α-lactalbumin polypeptide.

In some embodiments, provided nucleic acids are incorporated in a carrier or delivery vector. Useful delivery vectors include but are not limited to biodegradable microcapsules, immuno-stimulating complexes (ISCOMs), liposomes, and genetically engineered attenuated live carriers such as viruses or bacteria.

Amounts

In some embodiments, the amount of the composition is effective to induce or enhance an immune response against α-lactalbumin.

In some embodiments, the immune response comprises T-cells specific for α-lactalbumin. For example, the immune response may comprise CD4+ T-cells, CD8+ T-cells, or both CD4+and CD8+ T-cells. In some embodiments, the T-cells produce intereron gamma (IFNγ).

In some embodiments, the immune response comprises more than one type of T-cell response, e.g., at least two of Type-1 (IFNγ-producing proinflammatory T cells), Type-2 (IL-4, IL-5, and IL-13-producing regulatory T cells), and Type-17 (IL-17-producing proinflammatory T cells). In some embodiments, the immune response comprises both a Type-1 and a Type-17 response.

In some embodiments, the immune response comprises immunoglobulin-expressing cells (e.g., B-cells) specific for α-lactalbumin.

Additional Agents

In some embodiments, compositions are vaccine compositions.

In some embodiments, the composition further comprises one or more of: an adjuvant, a pharmaceutically acceptable carrier, a stabilizing agent or an antibiotic.

In some embodiments, the composition is especially formulated for a specific administration route, e.g., a systemic administration route.

### III. Exemplfication

### Example 1

Described herein are embodiments to provide prophylactic pre-emptive immunity against the development of adult onset cancers (e.g., male breast cancer) not associated with any definitive etiopathogenic agent. Safe and effective pre-emptive immunity may be induced in cancer-free subjects by vaccination against immunodominant tissue-specific self-proteins that are ‘retired’ from expression in normal tissues as part of the normal aging process but are expressed in tumors that emerge with age. In some embodiments, primary immunoprevention and/or immunotherapy of adult onset cancers like breast cancer (including male breast cancers) comprises vaccination against “retired” tissue-specific self-proteins, i.e., proteins no longer normally expressed in a host that may have expression recur or begin with tumorigenesis.

### Introduction

Checkpoint inhibitor antagonism provides suppressed immunity against growing tumors. Adoptive T cell transfer using chimeric antigen receptor T cell (CAR-T) technology has drastically improved overall survival of patients with leukemias and lymphomas. Prophylactic cancer vaccines that target hepatitis B and human papilloma virus have been remarkably effective in preventing liver cancer and cervical carcinoma, respectively. However, cancer vaccines that target non-pathogen antigens have often provided modest or disappointing results particularly when used as stand-alone treatments. The high failure rate of therapeutic cancer vaccines in advanced clinical trials may be attributed to several factors including inappropriate trial design, poor patient selection, and inconsistency in patient management, but may also be due in part to the use of target antigens that induce inadequate immune responses and adjuvants that fail to orchestrate appropriate and effective proinflammatory tumor immunity. However, perhaps the single most important reason why cancer vaccines have provided disappointing results may simply be due to their predominant use in the treatment setting when the tumor has already had a substantial head start in growth rather than in a prophylactic setting prior to the appearance of the emerging tumor. Indeed, the enviable success of the entire childhood vaccination program is based on providing immunity before engagement of the disease-causing pathogen and certainly before the onset of clinical symptoms.

Despite the enormous success of prophylactic vaccination against pathogens, there is currently no adult vaccination comparable to childhood vaccination for providing pre-emptive immunity against adult onset diseases like breast cancer and ovarian cancer. With the exception of the annual influenza vaccine, all current recommendations for adult vaccination are for either primary vaccines not received during childhood or for booster vaccinations to maintain the immunity against pathogens already induced during childhood. Such recommended boosters currently include herpes zoster vaccination for those 50 years and older to prevent eruption of latent varicella (chickenpox) virus, pneumococcus vaccination for those 65 years and older, and tetanus/diphtheria vaccination every 10 years. Thus, official recommendations for adult vaccinations focus exclusively on providing immunity against pathogens with no plans to induce immunity against adult onset diseases that are not definitively associated with any disease-causing pathogens. Here we describe a strategy to provide safe and effective prophylactic pre-emptive immunity against the development of adult onset cancers not associated with any definitive etiopathogenic agent.

### Use of Retired Proteins as Immunogens for the Treatment or Prevention of Cancers

Safe and effective protection against the emergence of adult onset cancers can be achieved by inducing targeted immunity against tissue-specific self-proteins that are ‘retired’ from expression at autoimmunogenic levels in normal tissues as a result of the natural aging process but are expressed in emerging adult cancers. For example, many of the breast-specific proteins dedicated to the lactation process are no longer expressed after the child-bearing years and breastfeeding ends. Similarly, ovarian-specific proteins that control ovarian reserve and production of mature follicles decline dramatically in postmenopausal ovaries. If such proteins are expressed in emerging breast or ovarian tumors, then pre-emptive immunity against these tissue-specific self-proteins would be able to protect against the development of tumors without inducing autoimmune complications. Thus, retired self-proteins, and fragments, including antigenic fragments thereof, can be the usedfor inducing pre-emptive immunity against some of the most lethal tumors confronted with age like triple negative breast cancer (TNBC) and breast cancer in male patients

In some embodiments, antigens based on α-lactalbumin , i.e, a retired or not naturally expressed tissue-specific self-protein, is used to induce an immune response in a patient at risk of or having humanbreast cancer, including male breast cancer and TNBC.

Use of Retired Tissue-Specific Self-Proteins for Prophylaxis and/or Treatment of Adult Onset Cancers

### α-Lactalbumin

In some embodiments, the antigen or immunogen is based on human α-lactalbumin, a breast-specific lactation protein with a full-length sequence of 142 amino acids transcribed from 4 exons and having a molecular weight of 16.2 kDa. (See Table 1.)

In some embodiments, the antigen or immunogen is a splice variant or fragment of α-lactalbumin In some embodiments, the immunogen is a 123 amino acid truncated splice variant transcribed from 3 exons of the LALBA gene having a molecular weight of 14 kDa. Expression of α-lactalbumin in normal human tissues is confined to the breast parenchyma during third trimester pregnancy and during lactation. It is not expressed in non-lactating breast tissue, and it is not expressed in any other normal human tissues at any time.

### Triple Negative Breast Cancer (TNBC)

Breast cancers are categorized according to their expression of hormone receptors that mediate signaling for tumor growth including the estrogen receptor (ER+), the progesterone receptor (PR+), and the epidermal growth factor receptor 2 (HER2+). Tumors expressing any of these hormone receptors are amenable to receptor-targeted therapy in the adjuvant setting. However, breast cancers that do not express any of these receptors are referred to as triple negative breast cancers (TNBCs) and currently have no proven targeted therapy. Despite representing only about 12% of all breast cancers, TNBCs account for a disproportionate higher percentage of breast cancer deaths due to their aggressive growth without need for any hormone growth factors and without the availability of any specific targeted therapies. TNBCs are twice as likely to occur in African-American women and in younger premenopausal women, and approximately 70% of the breast tumors occurring in women with mutations in their BRCA1 genes are TNBC. Thus, there is a great unmet need for strategies capable of achieving better control of this most lethal form of breast cancer.

### α-Lactalbumin in TNBC

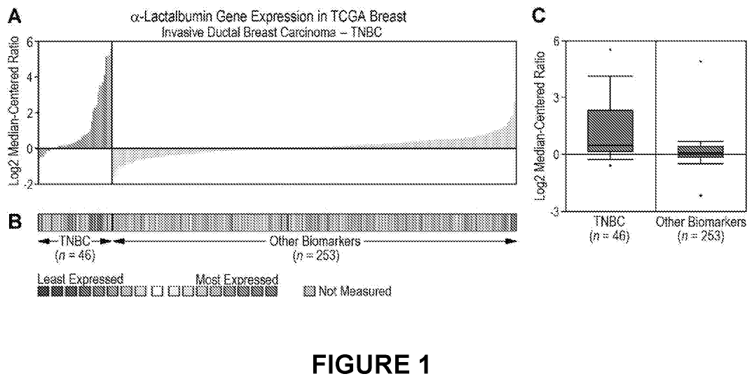

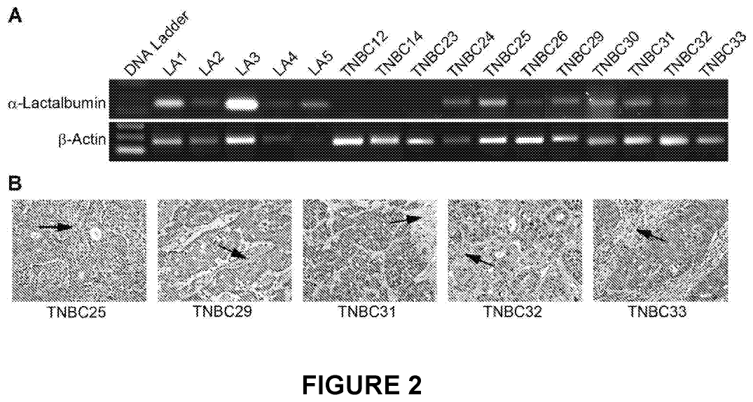

Although α-lactalbumin is not expressed in any non-lactating normal human tissues, we have found that α-lactalbumin shows significant increased expression in the majority of human TNBCs as determined by analysis of The Cancer Genome Atlas (TCGA) breast cancer database and shown as column charts (FIG. 1a), as a heat map (FIG. 1b), and as box charts (FIG. 1c). In addition, we have confirmed this gene expression pattern by performing RT-PCR on formalin-fixed paraffin embedded human TNBC tissues that showed expression of α- lactalbumin in 8/11 (72%) TNBCs comparable to expression levels occurring in benign human lactating adenomas (FIG. 2a). Our measured levels and observed incidence of α-lactalbumin gene expression in TNBC tissues were similar to those found in the TCGA breast cancer searchable database (FIG. 1). Moreover, immunohistochemical analysis showed that these gene expression levels correlated to detection of the protein in the mammary parenchyma of TNBC tumors (FIG. 2b).

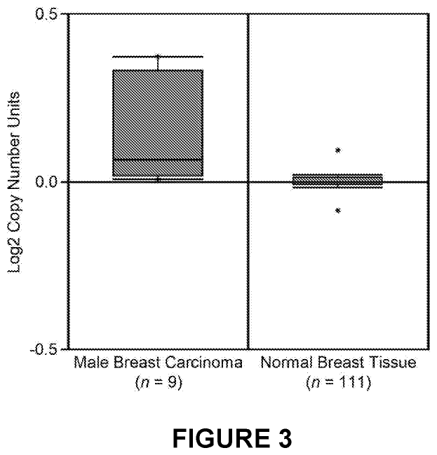

Breast tumors in males show significantly higher levels of α-lactalbumin gene expression compared to expression in normal breast tissue (P<0.02; FIG. 3). In any event, α-lactalbumin is expressed at high incidence in human TNBC tumors and perhaps in all male breast carcinomas and as such may serve as an effective immune target for TNBC immunoprevention and immunotherapy.

Some embodiments herein are directed to the methods for the prevention or treatment of male breast cancer comprising the use of an immunogen comprising a “retired” self-protein. Some embodiments herein are directed to the methods for the prevention or treatment of male breast cancer comprising the use of an immunogen comprising α-lactalbumin, or a fragment thereof.

Tumor Immunity Induced by Vaccination against α-Lactalbumin

The inventors' preclinical studies show that vaccination of mice against α-lactalbumin provided effective inhibition in the growth of both autochthonous and transplantable breast tumors. This tumor immunity was mediated by interferon-gamma (IFNγ) producing proinflammatory type 1 CD4+ and CD8+ T cells and occurred without any detectable autoimmune inflammatory damage to normal breast tissues and to all other normal tissues examined. Although this inhibition of tumor growth occurred when vaccination was administered using either prophylactic or therapeutic protocols, by far, the most dramatic results occurred when the immunity was induced prophylactically, thereby mimicking the way childhood vaccination against pathogens works so effectively by creating pre-emptive immunity prior to engagement with the pathogen.

### Clinical Trial Strategy for Immunoprevention of Adult Onset Cancers

### Triple Negative Breast Cancer (TNBC)

Women with mutations in their BRCA1 genes have more than a 60% risk of developing breast cancer in their lifetime and approximately 70% of the breast tumors they develop are TNBC. This high-risk group of women has the greatest need for a prophylactic TNBC vaccine because they develop an unusually high incidence of TNBC. Their only effective alternative is a traumatic life-changing event involving bilateral risk-reducing mastectomy (RRM) with immediate reconstruction to reduce disease risk without excessive disfigurement. Although about 50 such risk-reducing mastectomies (RRM) are currently performed each year at the Cleveland Clinic, RRM has not been widely accepted as the strategy of choice for reducing breast cancer risk by women at high risk for developing breast cancer. In two recent studies, 60% and 83% of cancer-unaffected women with BRCA1 or BRCA2 mutations chose surveillance over RRM to reduce their breast cancer risk. Thus, there is reason to believe that the majority of women at high risk for developing breast cancer would be available for recruitment into preventive vaccine clinical trials.

Most women electing RRM have mutations in their BRCA genes and/or an established family history of breast cancer. Consenting women will be vaccinated against α-lactalbumin several months prior to their voluntary mastectomy. After RRM, their surgically removed breast tissues will be examined extensively for signs of vaccine-induced autoimmune damage and their cellular and humoral immunity will be measured. Thus, this phase Ib trial will determine the safety of α-lactalbumin vaccination in healthy cancer-free women and will lay the groundwork for subsequent phase II/III trials designed to determine whether α-lactalbumin vaccination is effective in preventing TNBC in women at high genetic and/or familial risk for developing this form of breast cancer and eventually in any cancer-free women who voluntarily elect to be vaccinated.

### Example 2

### Prophylactic Inhibition of Male Breast Cancer in a Isograft Mouse Model

The prophylactic effects of methods of the present invention are demonstrated in a male isograft model of breast cancer (Nagasawa et al., “Mammary tumour induction by pituitary grafting in male mice: an animal model for male breast cancer.” Lab Anim. 1993 October; 27(4):358-63.).

6-8 week-old male mice are intravenously administered one to four doses of a composition comprising α-lactalbumin and an adjuvant, each dose approximately four weeks apart. Additional groups of mice may be used as controls or for comparison and may include, e.g., age- and strain-matched mice that are either not administered anything or are administered doses of: adjuvant only.

At 3-4 months of age, mice are grafted with isologous anterior pituitaries, 4 each, under the kidney capsules. Beginning at 5 months of age, each mouse is checked for palpable mammary tumors every 7 days until 12 months of age.

Tumor incidence, tumor volumes, and/or survival curves are compared between different mouse groups. Decreased tumor incidence, decreased tumor volume, and/or improved survival in mice receiving α-lactalbumin before inoculation with tumor cells demonstrates the prophylactic effects of the α-lactalbumin administration.

### Example 3

### Prophylactic Inhibition of Male Inflammatory Breast Cancer in a Xenograft Mouse Model

The prophylactic effects of methods of the present invention are demonstrated in a male model of canine and human inflammatory breast cancer (Caceres et al., “Steroid Tumor Environment in Male and Female Mice Model of Canine and Human Inflammatory Breast Cancer,” Biomed Res Int. 2016; 2016: 8909878).

6-8 week-old male mice are intravenously administered one to four doses of a composition comprising α-lactalbumin and an adjuvant, each dose approximately four weeks apart. Additional groups of mice may be used as controls or for comparison and may include, e.g., age- and strain-matched mice that are either not administered anything or are administered doses of: adjuvant only.

After the last dose of α-lactalbumin/adjuvant composition, mice are inoculated with a suspension of either 10⁶IPC-366 cells (a canine inflammatory mammary carcinoma cell line) or 10⁶ SUM149 (a human inflammatory breast cell line) by subcutaenous injection into the fourth inguinal mammary gland. Mice are inspected twice a week for the development of tumors. If tumors are detected in a mouse, the tumors are monitored weekly by palpitation and measured by calipers.

Tumor incidence, tumor volumes, and/or survival curves are compared between different mouse groups. Decreased tumor incidence, decreased tumor volume, and/or improved survival in mice receiving α-lactalbumin before inoculation with tumor cells demonstrates the prophylactic effects of the α-lactalbumin administration.

### Example 4

### Prophylactic Inhibition Of Male Breast Cancer in a Spontaneous Mammary Carcinoma Male Mouse Model

The prophylactic effects of methods of the present invention are demonstrated in a spontaneous mammary carcinoma male mouse model.

The wap-ras transgene is human c-Ha-ras gene regulated by the murine mammary-specific wap gene promoter. Line 69 of wap-ras mice have the wap-ras transgene inserted into the Y chromosome and spontaneously develop salivary tumors. In sub-line 69-2, developed after extensive inbreeding of line 69, males preferentially and spontaneously develop mammary adenocarcinomas tumors by 6 months after birth (Nielsen et al., “Histopathology of salivary and mammary gland tumors in transgenic mice expressing a human Ha-ras oncogene.” Cancer Res. 1991 Jul. 15; 51(14):3762-7).

6-8 week-old male mice of wap-ras subline 69-2 are intravenously administered one to four doses of a composition comprising α-lactalbumin and an adjuvant, each dose approximately four weeks apart. Additional groups of mice may be used as controls or for comparison and may include, e.g., age- and strain-matched mice that are either not administered anything or are administered doses of: adjuvant only.

After the last administration of α-lactalbumin, each mouse is checked weekly for palpable mammary tumors until death or until 12 months of age.

Tumor incidence, tumor volumes, and/or survival curves are compared between different mouse groups. Decreased tumor incidence, decreased tumor volume, and/or improved survival in mice receiving α-lactalbumin before inoculation with tumor cells demonstrates the prophylactic effects of the α-lactalbumin administration.

1. A method comprising the step of:
administering, to a mammalian male subject, a composition comprising an α-lactalbumin polypeptide or an immunogenic fragment thereof.

2. The method of claim 1, wherein the mammalian male subject is identified as being at risk of developing breast cancer.

3. The method of claim 1, wherein the mammalian male subject has one or more risk factors selected from the group consisting of older age, family history of breast cancer, high estrogen level, exposure to estrogen, lower androgen level, Klinefelter's syndrome, liver disease, obesity, testicle disease or surgery, gynecomastia, prolactinoma, radiation exposure to the chest, a genotype associated with breast cancer, or a gene expression profile associated with breast cancer.

4. The method of claim 1, wherein the mammalian male subject has one or more signs or symptoms of breast cancer.

5. The method of claim 1, wherein the mammalian male subject is diagnosed with breast cancer.

6. (canceled)

7. The method of claim 1, wherein the breast carcinoma expresses α-lactalbumin.

8. The method of claim 7, wherein the breast carcinoma exhibits increased expression of α-lactalbumin as compared to a reference level.

9. The method of claim 8, wherein the increased expression is at least 2-fold higher than the reference level.

10. (canceled)

11. The method of claim 9, wherein the increased expression is increased by at least 10-fold higher than the reference level.

12. The method of claim 1, wherein the mammalian male subject is a human.

13. The method of claim 1, wherein the composition comprises an amount of an α-lactalbumin polypeptide or an immunogenic fragment thereof effective to induce an immune response against α-lactalbumin.

14. The method of claim 13, wherein the immune response comprises T-cells specific for α-lactalbumin.

15. The method of claim 14, wherein the T-cells produce interferon gamma (IFNγ).

16. The method of claim 14 or 15, wherein the T-cells comprise CD4+ T-cells.

17. The method of claim 14 or 15, wherein the T-cells comprise CD8+ T-cells

18. (canceled)

19. The method of claim 1, wherein the immune response comprises immunoglobulin-expressing cells specific for α-lactalbumin.

20. The method of claim 19, wherein the immunoglobulin-expressing cells comprise B-cells.

21. The method of claim 1, wherein the composition further comprises an adjuvant.

22. The method of claim 1, wherein the α-lactalbumin is human α-lactalbumin.

23. The method of claim 1, wherein the composition is administered systemically.

24. (canceled)
